(19) United States
(12) Patent Application Publication
(10) Pub. No.: US 2004/0258630 A1
(43) Pub. Date: Dec. 23, 2004

(54) Antiplaque breath freshening consumable film

(76) Inventors: Thomas J. Boyd, Somerset, NJ (US); Guofeng Xu, Princeton, NJ (US); Abdul Gaffar, Princeton, NJ (US); David B. Viscio, Monmouth Jct., NJ (US)

Correspondence Address: Colgate-Palmolive Company, 909 River Road, P.O. Box 1343, Piscataway, NJ, 08855-1343, US

(21) Appl. No.: 10/601,473

(22) Filed: Jun. 23, 2003

(51) Int. Cl. A61K 7/16; A61K 7/22

(52) U.S. Cl. 424/49

(57) ABSTRACT

An orally consumable film composition for delivering antiplaque and breath freshening benefits to the oral cavity which is rapidly dissolvable or dispersible in the oral cavity, the composition being comprised of a homogeneous mixture of a water soluble or dispersible film forming polymer and an antibacterial ester having the formula

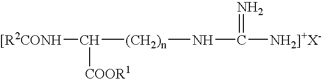

where R¹ is an alkyl chain of 1 to 8 carbon atoms, and R² is an alkyl chain of 6 to 30 carbon atoms and X is an anion.

### BACKGROUND OF THE INVENTION

1. Field of the Invention

The present invention relates to an orally consumable film for delivering antibacterial agents to the oral cavity and in particular a consumable film having antiplaque and breath freshening properties enhanced by the presence of an antibacterial ester compound incorporated in the film.

2. The Prior Art

Halitosis, the technical term for breath malodor, is an undesirable condition. Breath malodor results when proteins, particles from food, and saliva debris are decomposed by mouth bacteria. The tongue, with its fissures and large, bumpy surface area, retains considerable quantities of food and debris that support and house a large bacterial population. Under low oxygen conditions, the bacteria form malodorous volatile sulfur compounds (VSC) such as hydrogen sulfide and methyl mercaptans.

Dental plaque is a soft deposit which forms on teeth and is comprised of an accumulation of bacterial and bacterial by-products. Plaque adheres tenaciously at the points of irregularity or discontinuity, e.g., on rough calculus surfaces, at the gum line and the like. Besides being unsightly, plaque is implicated in the occurrence of gingivitis and other forms of periodontal disease.

A wide variety of antibacterial agents have been suggested in the art to retard breath malodor and plaque formation and the oral infections associated therewith. For example, halogenated hydroxydiphenyl ether compounds such as Triclosan are well known to the art for their antibacterial activity and have been used in oral compositions such as toothpastes to counter breath malodor and plaque formation by bacterial accumulation in the oral cavity.

Br. 1,352,420 discloses that the mono-N-higher aliphatic acyl arginine derivative adhere to the mucosa in the oral cavity and possess an antibacterial activity against oral bacterium such as Lactobacillus, a main pathogen of dental caries and bacterium belonging to the genus Staphylococcus, a main pathogen of alveolar pyorrhea.

U.S. Pat. No. 5,874,068 discloses an antiplaque effective mouthrinse containing a N^{α}-acyl acidic amino acid ester salt, the salt being stabilized by the presence in the mouthrinse of a monohydric alcohol such as ethanol, as aqueous compositions containing these salts normally undergo hydrolysis in aqueous environments.

It is known to the art to use consumable water soluble or dispersible films adapted to disintegrate in the oral cavity which films contain flavoring agents for delivering breath freshening agents to mask or reduce bacteria caused breath malodor. For example, U.S. Pat. No. 6,419,903 discloses a consumable breath freshening film adapted to dissolve in the mouth of the user, the film being comprised of a water soluble hydroxyalkylmethyl cellulose, a water dispersible starch and a flavoring agent.

U.S. Pat. No. 6,177,096 discloses a film composition containing therapeutic and/or breath freshening agents for use in the oral cavity prepared from a water soluble polymer such as hydroxypropylmethyl cellulose, hydroxypropylcellulose and a polyalcohol such as glycerol, polyethylene glycol.

Although the prior art water soluble consumable films have provided breath freshening benefits, the art continually seeks to enhance such benefits.

### SUMMARY OF THE INVENTION

In accordance with the present invention there is provided orally consumable film composition effective to reduce breath malodor and plaque formation on teeth having incorporated in the film matrix an antibacterial ester and salts thereof having the formula

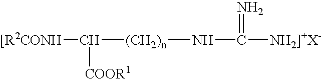

where R¹ is an alkyl chain of 1 to 8 carbon atoms, and R² is an alkyl chain of 6 to 30 carbon atoms and X is an anion,

### DETAILED DESCRIPTION OF THE INVENTION

The film of the present invention comprises a consumable water soluble or dispersible forming polymer containing an antibacterial ester compound namely, a Na-acyl acidic amino acid ester compound. The film can further comprise water, flavor agents, plasticizing agents, emulsifying agents, coloring agents, sweeteners and other compatible antibacterial and other therapeutic agents.

Antibacterial Ester

In the above identified antibacterial ester formula, R²CO may be a natural system mixed fatty acid residue such as coconut oil fatty acid tallow fatty acid residue and the like, or a mono-fatty acid residue such as lauroyl, myristyl, stearoyl and the like, the lauroyl group being preferred.

Examples of antibacterial ester salts of the above identified formula include an inorganic acid salt such as hydrochloride, sulfate or an organic salt such as acetate, tautarate or citrate, the chloride salt being preferred.

Examples of antibacterial ester compounds preferred in the practice of the present invention are antibacterial ester compound of the above-identified formula wherein n in the formula equals 3 as for example, N^{α}-cocoyl-L-arginine methyl ester, N^{α}-cocoyl-L-arginine ethyl ester, N^{α}-cocoyl-L-arginine propyl ester, N^{α} stearoyl-L-arginine methyl ester, N^{α} steoryl-L-arginine ethyl ester hydrochloride. The term “cocoyl” is an abbreviation for coconut oil fatty acid residue, and chloride salts of these compounds, these ester compounds hereinafter being referred to as arginine derivative compounds. A preferred arginine derivative compound is the hydrochloride salt of ethyl lauroyl arginine.

The antibacterial ester of the present invention is present in the film compositions of the present invention at a concentration of about 0.05 to about 25% by weight and preferably about 0.075 to about 20% by weight.

Arginine derivative compounds and their salts in particular show excellent inhibitory effect against microorganisms which possess relatively strong resistance to bacterium such as S.aureus, S.mutans, F.nucleatum which are involved in plaque formation on teeth. As will hereinafter be demonstrated, the plaque inhibitory effect of the film composition of the present invention is comparable to that of Triclosan, the only antibacterial agent approved by the U.S. Federal Drug Administration for use in oral care formulations.

Film Matrix

Water soluble or dispersible film forming agents used to form the film matrix of the present invention include water soluble polymers such as polyvinyl pyrrolidone, hydroxyethyl cellulose, hydroxypropyl methyl cellulose, hydroxyalkyl celluloses such as hydroxypropyl cellulose, carboxymethyl cellulose, polyvinyl alcohol, sodium alginate, alginate esters, guar gum, xanthan gum, gelatin, polyethylene oxide, polyethylene glycol, carrageenan, pullulan, locust bean gum as well as water dispersible polymers such as polyacrylates, carboxyvinyl copolymers, methyl methacrylate copolymers and polyacrylic acid. A low viscosity hydropropylmethyl cellulose polymer (HPMC) having a viscosity in the range of about 1 to about 40 millipascal seconds (mPa.s) as determined as a 2% by weight aqueous solution of the HPMC at 20° C. using a Ubbelohde tube viscometer is a preferred film matrix material as is disclosed in U.S. Pat. No. 6,419,903, the disclosure of which is herein incorporated by reference. Preferably the HPMC has a viscosity of about 3 to about 20 mPa-s at 20° C. such HMPC is available commercially from the Dow Chemical Company under the trade designation Methocel E5 Premium LV. Methocel E5 Premium LV is a USP grade, low viscosity HPMC having 29.1% methoxyl groups and 9% hydroxyproxyl group substitution. It is white or off-white free flowing dry powder. As a 2 weight % solution in water as measured with Ubbelohde tube viscometer it has a viscosity of 5.1 to mPa-s at 20° C.

The hydroxyalkyl methyl cellulose is incorporated in the film composition in amounts ranging from about 10 to about 60% by weight and preferably about 15 to about 40% by weight. Cold water dispersible, swellable, physically modified and pregelatinized starches are particularly useful as texture modifier to increase the stiffness of the hydroxyalkyl methyl cellulose polymer films of the present invention. To prepare such starch products, the granular starch is cooked in the presence of water and possibly an organic solvent at a temperature not higher than 10° C. higher than the gelatinization temperature. The obtained starch is then dried.

Pregelatinized corn starch is available commercially. A preferred starch is available under the trade designation Cerestar Polar Tex-Instant 12640 from the Cerestar Company. This Cerestar starch is a pregelaterized, stabilized and crosslinked waxy maize starch. It is readily dispersible and swellable in cold water. In its dry form, it is a white free flowing powder with an average particle size no greater than 180 micrometers and 85% of the particles are smaller than 75 micrometers. It has a bulk density of 441 bs/ft3.

The pregelatinized starch may be incorporated in the film matrix of the present invention in an amount ranging from about 5 to about 50% by weight and preferably about 10 to about 35% by weight.

Emulsifiers

Emulsifying agents are incorporated in the film matrix ingredients to promote homogeneous dispersion of the ingredients. Examples of suitable emulsifiers include condensation products of ethylene oxide with fatty acids, fatty alcohols, polyhyrric alcohols (e.g., sorbitan monostearate, sorbitan oleate), alkyl phenols (e.g., Tergitol) and polypropyleneoxide or polyoxybutylene (e.g., Pluronics); amine oxides such as dimethyl cocamine oxide, dimethyl lauryl amine oxide and cocoalkyldimethyl amine oxide polysorbates such as Tween 20, Tween 40 and Tween 80 (Hercules), glyceryl esters of fatty acid (e.g., Arlacel 186), natural and synthetic lipids such as lecithin. The emulsifying agent is incorporated in the film matrix composition of the present invention at a concentration of about 0.01 to about 10% by weight and preferably about 0.1 to 5.0% by weight.

Flavor Agents

Flavor agents that can be used to prepare the film of the present invention include those known to the art, such as natural and artificial flavors. These flavor agents may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. These flavor agents can be used individually or in admixture. Commonly used flavor include mints such as peppermint, artificial vanilla, cinnamon derivatives, and various fruit flavors, whether employed individually or in admixture. The amount of flavoring agent employed is normally a matter of preference subject to such factors as flavor type, individual flavor, and strength desired.

Generally the flavor agent is incorporated in the film of the present invention in an amount ranging from about 0.1 to about 35% by weight and preferably about 3 to about 25% by weight.

Sweeteners useful in the practice of the present invention include both natural and artificial sweeteners. Suitable sweetener include water soluble sweetening agents such as monosaccharides, disaccharides and plysaccharides such as xylose, ribose, glucose (dextrose), mannose, glatose, fructose (levulose), sucrose (sugar), maltose, water soluble artificial sweeteners such as the soluble saccharin salts, i.e., sodium or calcium saccharin salts, cyclamate salts dipeptide based sweeteners, such a L-aspartic acid derived sweeteners, such as L-aspartyl-L-phenylalaine methyl ester (aspartame) and sucralose. In general, the effective amount of sweetener is utilized to provide the level of sweetness desired for a particular composition, will vary with the sweetener selected. This amount will normally be about 0.01% to about 2% by weight of the composition.

Plasticizers

Plasticizers are small molecules incorporated into the film matrix to modify or improve the mechanical properties of the film, such as elasticity and elongation. Examples of suitable plasticizers are, but not limited to, water, propylene glycol, ethylene glycol, glycerin, polyethylene glycol, triacetin and maltodextrin. These plasticizers can be used individually or in admixture. The plasticizers are incorporated in the film matrix composition of the present invention at a concentration of about 0.5% to about 30% by weight and preferably about 1% to 20% by weight.

Other Ingredients

The compositions of the present invention can also contain coloring agents or colorants. The coloring agents are used in amounts effective to produce the desired color and include natural food colors and dyes suitable for food, drug and cosmetic applications. These colorants are known as FD&C dyes and lakes. The materials acceptable for the foregoing spectrum of use are 20 preferably water-soluble, and include FD&C Blue No.2, which is the disodium salt of 5,5indigotindisulfonic acid. Similarly, the dye known as Green No.3 comprises a 15 triphenylmethane dye and is the monosodium salt of 4-[4-N-ethyl-p-sulfobenzylamino)diphenyl-methylene]-[1-N-ethyl-N-sulfonium benzyl)-2,5-cyclo-hexadienimine]. A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.

Antibacterial agents compatible with the antibacterial ester compound may also be included in the film matrix of the present invention, such antibacterial agents including Triclosan, cetylpyridinium chloride, chlorhexidene, natural herbs such as Magnolia, metal salts such as stannous chloride, stannous citrate and stannous gluconate and zinc salts such as zinc chloride, zinc citrate and zinc gluconate, and copper salts such as copper gluconate.

Film Manufacture

In preparing the film composition according to the present invention, a water soluble or water dispersible film forming agent such as hydroxyalkylmethyl cellulose is dissolved in a compatible solvent such as water heated to about 60° C. to about 95° C. to form a film forming composition. Thereafter, there is optionally added in the sequence, a second film forming agent such as starch, sweetener, surfactant, flavor, antibacterial ester and other antiplaque agents to prepare a film ingredient slurry.

The slurry is cast on a releasable carrier and dried. The carrier material must have a surface tension which allows the film solution to spread evenly across the intended carrier width without soaking to form a destructive bond between the film and the carrier substrate. Examples of suitable carrier materials include glass, stainless steel, Teflon and polyethylene impregnated paper. Drying of the film may be carried out at elevated temperatures in a convection oven or by transversing through a zoned dryer at approximately 10-100 inches/min at temperatures ranging for example from, 70° C. to 120° C., using a drying oven, drying terminal, vacuum drier, or any other suitable drying equipment for residence times which do not adversely effect the ingredients of which the film is composed.

The film once formed is segmented into dosage units by die-cutting or slitting-and-die cutting. The segmented film has a strip width and length corresponding to about the size of a postage stamp, generally about 12 to about 30 millimeter in width and about 20 to about 50 millimeters in length. The film has a thickness ranging from about 15 to about 80 micrometers, and preferably about 40 to 60 micrometers.

The following examples further describe and demonstrate preferred embodiments within the scope of the present invention. The examples are given solely for illustration, and are not to be construed as limitation of this invention as many variations thereof are possible without departing from its spirit and scope.

### EXAMPLE

A series of films containing varying amounts of the arginine derivative compound the hydrochloride salt of ethyl lauroyl arginine designated Compositions A, B and C were prepared by using the ingredients listed in Table I below. In preparing the film, the hydroxy propylmethylcellulose polymer ingredient (Methocel E5LV) and carrageenan was added at a temperature of 70° C. to 90° C., to half the amount of total deionized water used, and the solution stirred for 20 minutes at a slow speed using a IKA Labortechnik Model RW20DZMixer. The remaining amount of water maintained at room temperature (21° C.) was then added and the mixing continued for 40 minutes. To this solution was added the corn starch ingredient (Cerestar Polar Tex Instant 12640) and the mixture stirred for an additional 20 minutes until the starch was completely dispersed and a homogeneous mixture was formed. To this mixture was added sucralose and mixed for 10 minutes after which the emulsifier Tween 80 was added and mixed for an additional 5 minutes. Thereafter, flavor was thoroughly mixed for an additional 30 minutes to form a slurry emulsion to which as a final step the hydrochloride salt of ethyl lauroyl arginine HCl (ELAH) dispersed in canola oil was slowly added until evenly dispersed in the film ingredient slurry. The emulsion was then cast on a polyethylene coated paper substrate and dried in a convection oven at 110° C. to form a solid thin (30-60 μm thick) film.

For purposes of comparison, the procedure of Example I was repeated to prepare a film composition designated Composition D with the exception that no ethyl lauroyl arginine HCl was incorporated in the film composition.

TABLE 1

Composition Wt. %

| Ingredients | A | B | C | D |
|---|---|---|---|---|
| HPMC | 41.0 | 41.0 | 38 | 41.0 |
| Carageenan | 0.50 | 0.50 | 0.50 | 0.50 |
| Corn starch | 19.0 | 19.0 | 17 | 19.0 |
| Flavor | 25.0 | 25.0 | 18 | 25.0 |
| Tween 80 | 2.30 | 2.30 | 2.1 | 2.30 |
| Canola oil | 4.50 | 4.50 | 4.1 | 4.50 |
| Sucralose | 1.4 | 1.4 | 1.3 | 1.4 |
| Propylene glycol | 1.25 | 6.25 | 11.5 | 0 |
| ELAH | 0.50 | 2.5 | 5.0 | 0 |
| Water | Q.S. | Q.S. | Q.S. | Q.S. |

The antiplaque activity of Compositions A, B, C and D was assessed using a flow cell model of the type disclosed in the Journal of Dental Research, vol. 73(11), pp. 1748-1755 (1994) using human saliva as the bacterial source and single crystal germanium prisms as the oral surface model. After pretreatment of these surfaces with a precisely cut strip (10 mm×20 mm), they were rinsed with artificial saliva (1 part porcine mucin 25 g/L, and 1 part saliva buffer solution) prior to exposure to bacteria, and exposed to treatment in the flow cell. The plaque index of the deposits on the prisms was determined by infrared spectrophotometry.

Plaque Score

Compositions A, B and C were assessed for overall plaque inhibition versus the comparative Composition D which did not contain an antibacterial agent which was simultaneously run in the system. The lower the Plaque Index the more effective the antiplaque agent. The results recorded in Table II below show a 30-40% reduction in plaque effected by Film Compositions A, B and C when compared to Comparative Film Composition D.

TABLE II

| Film Composition | Plaque Index | % Reduction |
|---|---|---|
| A | 0.429 | 37.7 |
| B | 0.466 | 32.4 |
| C | 0.486 | 29.6 |
| D | 0.690 | — |

### Example II

A second series of film compositions designated E and F were prepared following the procedure of Example I, in which Composition E contained 5% by weight (dry film) ELAH, Composition F contained 5% by weight (dry film) ELAH and 1.5% by weight (dry film) zinc gluconate. For purposes of comparison, film Composition G prepared in the same manner as Film A but which contained no ELAH and Film Composition H, a commercially available breath freshening film were tested for antiplaque efficacy in the artificial mouth test model. The tests were run in parallel under identical conditions wherein 4 hydroxyapatitie discs (HAP) disks were coated with pellicle for two hours followed by additional 2 hours of bacteria attachment. The disks were mounted in a flow cell and 10 μmL solution of film (containing 150 mg film) were then passed over the surface of the disks for 1-2 minutes; water was passed over the disks for 10 minutes to wash. The flow cell was then connected to the artificial mouth chemostat circulator and incubated for 8-12 hours. The procedure was repeated 4 times, and thereafter the HAP disks were dismounted and bacteria on the disks were detached. The bacteria were quantified by optical density readings. The results of this test procedure are recorded in Table III below.

TABLE III
Optical Density

| Film Composition | Mean | Standard Deviation | % Reduction |
|---|---|---|---|
| E | 0.23 | 0.02 | 31.2 |
| F | 0.20 | 0.03 | 38.9 |
| G | 0.33 | 0.05 | 0 |
| H | 0.38 | 0.05 | 4.0 |

The results recorded in Table III show that antibacterial films of the present invention (Films E, F) effect a significant reduction in antiplaque formation when compared to films G, H that did not contain the arginine derivative compound.

### Example III

The procedure of Example II was repeated in which a series of film compositions designated J, K were prepared following the procedure of Example I in which Composition E contained 5% by weight (dry film) ELAH, Composition L contained 5% by weight (dry film) ELAH and 1.5% by weight (dry film) zinc gluconate. For purposes of comparison, Composition M contained 5% by weight (dry film) Triclosan, but no ELAH and Composition H was a placebo containing no ELAH or antibacterial ester compound.

The antiplaque efficacy of the films was evaluated following the artificial mouth model described in Example II. The results of these tests are recorded in Table IV below.

TABLE IV
Optical Density

| Film Composition | Mean | Standard Deviation | % Reduction |
|---|---|---|---|
| J | 0.23 | 0.02 | 31.2 |
| K | 0.20 | 0.03 | 38.9 |
| L | 0.23 | 0.03 | 30.6 |
| M | 0.33 | 0.05 | 0.0 |

The results recorded in Table IV indicate that ELAH is at least effective as Triclosan in reducing plaque formation when delivered to the oral cavity from a consumable film and that a combination of ELAH and a metal salt such as zinc gluconate provides antiplaque efficacy superior to Triclosan.

### Example IV

A series of film compositions designated Compositions N, P, Q were prepared following the procedure of Example I, in which Composition N contained 0.50 by weight ELAH, Composition P contained 2.5% ELAH and Composition Q contained 5% by weight ELAH.

For purposes of comparison film Composition R was also prepared following the procedure of Example I except that no ELAH was incorporated in the film composition.

Film Compositions N, P, Q and R were evaluated for breath freshening efficacy by an in-vitro volatile sulfur compound (VSC) reduction assay. In this assay a known amount of film is dissolved in 3.0 milliliters (ml) of saliva in a glass vial. After incubation at 37° C. overnight, the headspace of the solution is sampled and analyzed for the VSC. The VSC assay results are presented in Table V below.

TABLE V
VSC in the headspace

| Film Composition | Baseline | After 24 hours | VSC Reduction (%) |
|---|---|---|---|
| N | 27.3 | 23.90 | 12.5 |
| O | 27.3 | 18.36 | 32.8 |
| Q | 27.3 | 4.56 | 83.3 |
| R | 27.3 | 25.61 | 6.3 |

The VSC assay results recorded in Table V demonstrate the increase in VSC reduction as the concentration of the antibacterial ester ELAH in the film matrix is increased.

What is claimed is:

1. An orally consumable film composition for delivering antiplaque and breath freshening agents to the oral cavity which rapidly dissolves or disintegrates when applied in the oral cavity, the composition being comprised of a homogeneous mixture of a water soluble or dispersible film forming polymer and an antibacterial ester having the formula 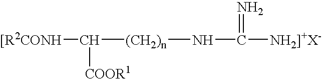
where R¹ is an alkyl chain of 1 to 8 carbon atoms, and R² is an alkyl chain of 6 to 30 carbon atoms and X is an anion.

2. The film composition of claim 1 wherein the polymer is an hydroxyalkyl cellulose.

3. The film composition of claim 2 wherein the hydroxyalkylcellulose is hydroxymethylpropyl cellulose.

4. The film composition of claim 1 wherein the water dispersible polymer is present at a concentration of about 10 to about 60% by weight.

5. The film composition of claim 1 wherein the antibacterial ester is present in the film at a concentration of about 0.05 to about 25% by weight.

6. The film composition of claim 1 the antibacterial is an arginine derivative compound.

7. The film composition of claim 1 wherein the arginine derivative compound is the hydrochloride salt of ethyl lauoryl arginine.

8. The film composition of claim 1 wherein the film also contains an antiplaque metal salt.

9. The film composition of claim 8 wherein the metal salt is zinc gluconate.
